(19) United States
(12) Patent Application Publication
(10) Pub. No.: US 2017/0035600 A1
(43) Pub. Date: Feb. 9, 2017

(54) DISPOSABLE URINARY FUNNEL

(71) Applicant: Juan Preciado, Chandler, AZ (US)

(72) Inventor: Juan Preciado, Chandler, AZ (US)

(21) Appl. No.: 14/818,847

(22) Filed: Aug. 5, 2015

(51) Int. Cl. A61F 5/453; E03D 13/00; A61F 5/455

(52) U.S. Cl. CPC A61F 5/453; A61F 5/4556; E03D 13/005

(57) ABSTRACT

A disposable urinary funnel for assisting a male user to prevent splattering of urine stream, the urinary funnel comprises a foldable, elongate tubular housing with an inlet at a first open end and an outlet at a second open end. The urinary funnel further comprises at least two loops attached externally to sidewalls of the first open end, for holding the funnel by placingfingers into the loop and positioning the inlet around the male reproductive organ while urinating from a standing position.

### FIELD OF THE INVENTION

The present invention generally relates to a disposable urinary device and more specifically relates to a biodegradable urinary funnel configured for use by male users to prevent splattering of urine steam on or around a toilet.

### BACKGROUND

Generally a toilet or similar urinal facilities are designed to receive urine stream from male users in a standing position. In the process, however, urine stream splatters on the commode and any surrounding areas around the toilet, no matter how careful the male users may be. The splattered urine not only soils the commode but also soils the users dress, foot wear and splattered urine droplets may also contaminate exposed skin of the users. Therefore, urine splatter results in a non-hygienic condition in toilet facilities and requires additional sanitation of the commode and around the toilet frequently.

In addition, unclean restrooms or toilets such as public or residential restrooms further necessitates users to urinate from standing position. In order to avoid the unintentional splattering of urine, a male user would otherwise need to sit on the commode seat which can be very time consuming, inconvenient, and in certain circumstances uncomfortable and unhygienic based on the sanitary condition of the commode seat. Besides contamination, the stench of splattered urine further adds to the discomfort of users.

There are several patents which disclose various urinary assistance devices, most of the devices are complex in construction which are difficult to store, deploy and dispose safely after use. Therefore, there still exists a need for a biodegradable urinary funnel that is easily deployable and safely disposable by flushing in the toilet.

### SUMMARY

The present invention relates to a disposable urinary assisting device or a urinary funnel, comprising an elongate tubular housing made of a light weight, foldable biodegradable material. The housing comprises an inlet at a first open end and an outlet at a second open end. The housing further comprises at least two loops attached externally to sidewalls of the first open end, for holding the funnel by a male user while urinating from a standing position. The urinary funnel of the present invention is configured for use by male users to prevent splattering of urine stream on or around the toilet. The urinary funnel allows male users to direct the urine stream directly into the designated area of the toilet in a hygienicway without splash back or splattering.

In an embodiment, the disposable urinary funnel is flushable into a toilet. The urinary funnel is made of a light weight, biodegradable material that is foldable for easy storage and transport prior to use. In an embodiment, the disposable urinary funnel comprises a flat folded structure, the inlet can be expanded to form an opening by inserting thumb and index finger into the loops and gently pulling apart the side walls of the housing at the inlet. The inlet at the first open end is configured to be placed around the reproductive organ during urination, to prevent splattering of urine stream and to direct the urine stream into the commode.

### BRIEF DESCRIPTION OF DRAWINGS

FIG. 1 illustrates a side view of the disposable urinary funnel according to an embodiment of the present invention.

FIG. 2 illustrates a side perspective view of the disposable urinary funnel held by a user.

FIG. 3 illustrates a front perspective view of the disposable urinary funnel held by the user.

### DETAILED DESCRIPTION OF DIFFERENT EMBODIMENTS

In the following description, for the purposes of explanation, specific construction details, arrangements, and materials are set forth in order to provide a more thorough understanding of the present invention. It will be apparent to those skilled in the art, however, that the present invention may be practiced without these specific details. In other instances, well known manufacturing methods and structures have not been described in detail so as not to obscure the present invention unnecessarily.

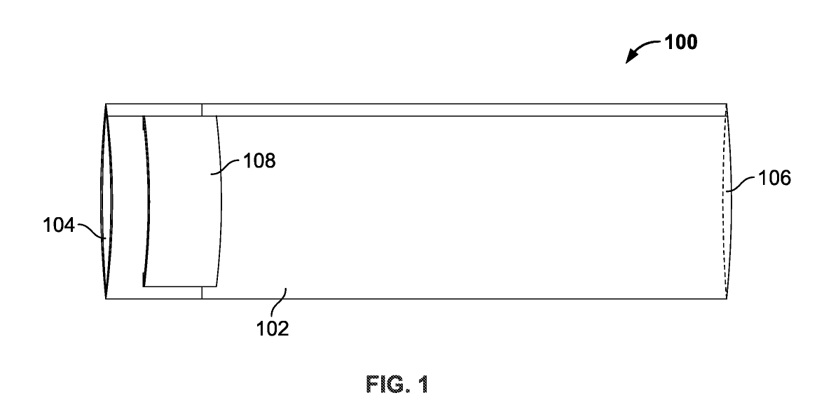

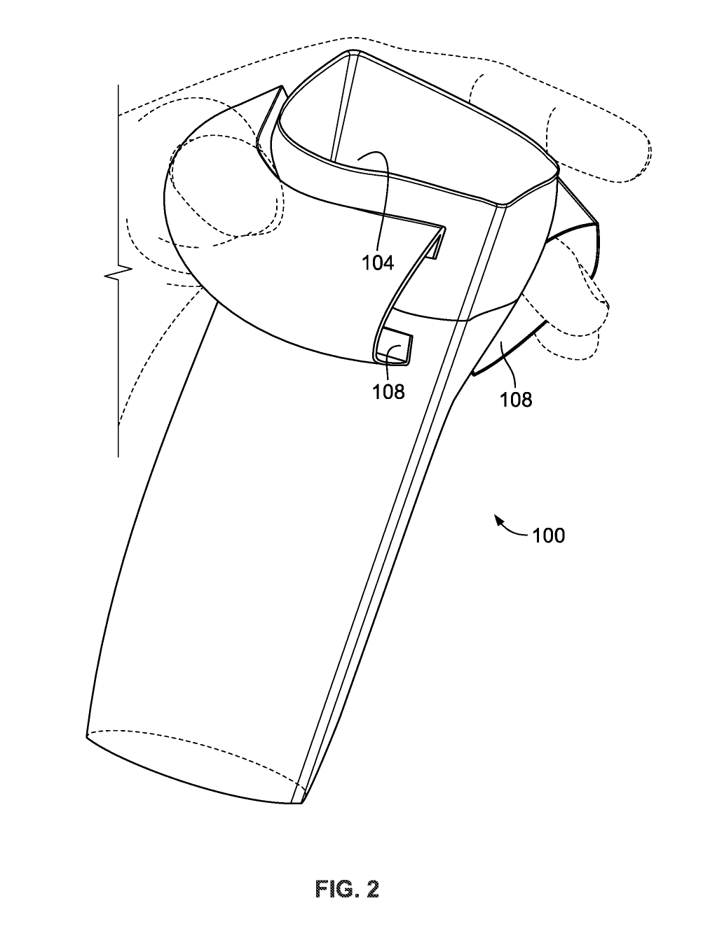

Referring to FIG. 1 illustrating a side view of the disposable urinary funnel 100, comprising an elongate tubular housing 102 made of a biodegradable material. The housing 102 comprises an inlet 104 at a first open end and an outlet 106 at a second open end. The housing 102 further comprises at least two loops 108 attached externally to sidewalls of the first open end or the inlet portion 104. The loops 108 are configured to be held using fingers of a male user while urinating from a standing position. For example, the user can place his thumb finger and index finger into the loops 108 near the inlet 104, for holding the funnel 100 as shown in FIG. 2, which illustrates a side perspective view of the urinary funnel 100.

The urinary funnel 100 of the present invention is configured for use by male users to prevent splattering of urine stream on or around the toilet. In other words, the urinary funnel 100 allows male users to direct the urine stream into the designated area of the toilet in a hygienic way without splash back or splattering. The inlet 104 at the first open end is configured to be placed around the reproductive organ during urination, to prevent splattering of urine stream and to direct the urine stream directly into the water reservoir area of the commode.

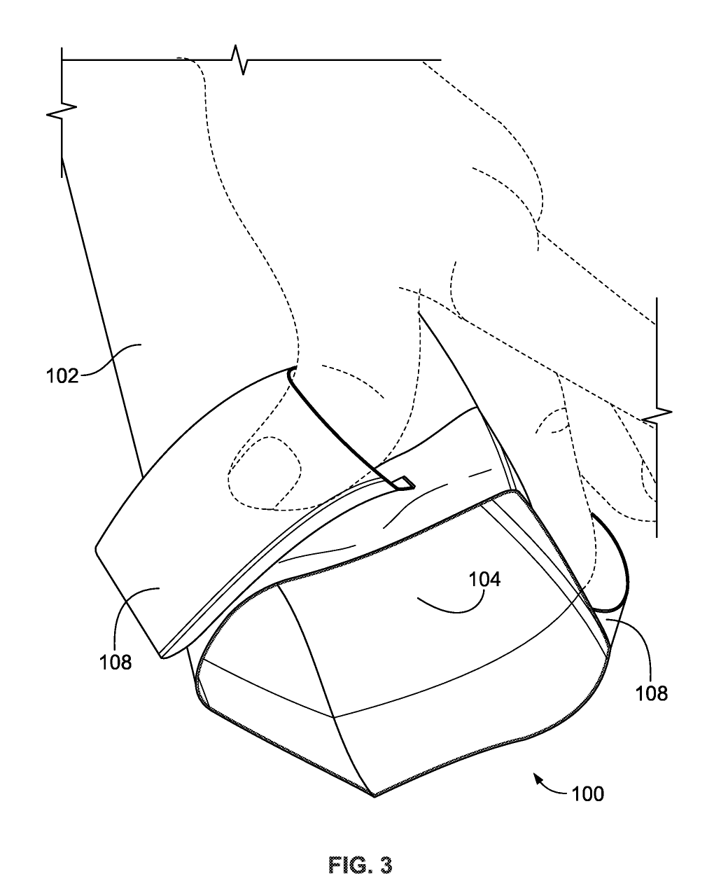

In an embodiment, the urinary funnel 100 is disposable by flushing away in toilet. The urinary funnel 100 is made of a light weight, biodegradable material that is foldable for easy storage and transport, prior to use. In an embodiment, the disposable urinary funnel 100 comprises a flat folded structure, the inlet portion 104 can be expanded to form an opening by inserting thumb and index finger into the loops 108 and gently pulling apart the side walls of the first open end of the housing 102, as shown in FIG. 3.

The urinary funnel 100 is made from a biodegradable material that is able to withstand variations in pressure and flow conditions of the urine stream. The biodegradable material is selected in such a way that the material will not clog the drain when the urinary funnel 100 is flushed into the toilet after use.

The urinary funnel 100 is easy to use and offers many advantages such as modifiable length and simple design, which allows users of all ages to easily, comfortably and confidently use the urinary funnel 100 without any extra effort and helps to maintaining a clean restroom. It also helps to avoid the unpleasant and lingering stench of splattered urine and allows users the convenience of urinating without having to raise the toilet seat, and still avoiding any urine from splashing or spilling accidentally on the user or any other unintended area or surface. The urinary funnel is safe for use with any commode and septic system.

It will be apparent to those skilled in the art that the foregoing description is for illustrative purposes only, and that various changes and modifications can be made to the present invention without departing from the overall spirit and scope of the present invention. The full extent of the present invention is defined and limited only by the following claims.

What is claimed is:

1. A disposable urinary funnel comprising:
an elongate tubular housing made of a biodegradable material, comprises an inlet at a first open end and anoutlet at a second open end; and
at least two loops attached externally to sidewalls of the first open end, for holding the funnel by a male user while urinating from a standing position.

2. The disposable urinary funnel of claim 1, is flushable into a toilet.

3. The disposable urinary funnel of claim 1, wherein the two loops are configured to receive at least two fingers of the user for holding the funnel.

4. The disposable urinary funnel of claim 1, wherein the inlet is expanded by gently pulling apart the sidewalls of the first open end.

5. The disposable urinary funnel of claim 1, is flat-foldable for storage and transport.

6. The disposable urinary funnel of claim 1, wherein the inlet is configured to be positioned around a male reproductive organ during urination.

7. The disposable urinary funnel of claim 1, wherein the outlet is configured to be positioned adjacent to the toilet.
